(19) United States
(12) Patent Application Publication
(10) Pub. No.: US 2014/0120065 A1
(43) Pub. Date: May 1, 2014

(54) PROTECTION FROM LETHAL IRRADIATION WITH MESENCHYMAL STROMAL CELLS

(71) Applicant: RADIOPROTECT ALPHAPTOSE UG (HAFTUNGSBESCHRÄNKT) & Co. KG, Eutin (DE)

(72) Inventors: Axel R. Zander, Lubeck (DE); Claudia Lange, Hamburg (DE); Christof Westenfelder, Salt Lake City, UT (US)

(21) Appl. No.: 13/976,622

(22) PCT Filed: Dec. 29, 2011
(86) PCT No.: PCT/EP2011/074232

§ 371 Date: Jan. 17, 2014

Related U.S. Application Data
(60) Provisional application No. 61/428,677, filed on Dec. 30, 2010.
(60) Provisional application No. 61/439,556, filed on Feb. 4, 2011.

(51) Int. Cl. A61K 35/28

(52) U.S. Cl. CPC A61K 35/28
USPC 424/93.7; 435/325

(57) ABSTRACT

The present invention relates to the use of mesenchymal stromal cells (MSCs) for the treatment of supralethal radiation injury.

### FIELD OF THE INVENTION

The present invention relates to the use of mesenchymal stromal cells (MSCs) for the treatment of supralethal radiation injury.

### BACKGROUND OF THE INVENTION

Radiation injury is caused by reactor accidents, atomic bombs, nuclear terrorism and other radiological emergencies. Depending on the dose of exposure it leads to various clinical manifestations from nausea and vomiting to severe gastrointestinal problems, bone marrow failure, and after extreme high doses to toxicity to the central nervous system.

Survival of radiation-induced bone marrow failure depends on the dose of radiation received and the intensity of supportive care which can protect from otherwise lethal infection and give surviving stem cells a chance to expand. To provide the best possible care for radiation accident victims in acts of terrorism or catastrophic incidences, medical countermeasures need to be made within the first few days for optimal efficacy. The “response category concept” proposed by Fliedner et al. (Fliedner T M, Friesecke I, Beyrer K (2001) Medical Management of Radiation Accidents: Manual on the Acute Radiation Syndrome. Oxford: British Institute of Radiology) evaluates the radiation induced tissue damage and rates the hematopoietic score 4-H4 (the highest score for hematopoietic damage) as case with little probability for autologous recovery, i.e. repopulation of the bone marrow by residual hematopoietic stem cells. Combined approaches including presenting symptoms, biomarkers and physical dosimetry are employed to categorize affected individuals for best medical countermeasures.

Overall measures include supportive care, treatment with growth factors within the first two weeks after radiation exposure, or hematopoietic stem cell transplantation (HSCT). Treatment options depend on the radiation dose to which the subject was exposed. Lower radiation doses can be overcome by supportive care, growth factor application and transfusions. At high doses bone marrow failure can be overcome with hematopoietic stem cell (HSC) transplantation if a suitable donor is available. However, HSC transplantation is time-consuming due to the search for a suitable donor and its outcome is uncertain.

Since radiation effects on blood stem cells occur at doses generally lower than on other critical organs, the rapidly emerging changes in the peripheral blood cell lineages dictate the treatment options. Animal and human studies indicate that hematopoietic pluripotent stem cells have a D0 of about 95cG. D0 is the dose increment that reduces the cell survival to 37%. In fact, total body exposure at doses more than 7-8 Gy total body irradiation (TBI) in human corresponds to medullar eradication and a hematopoietic score of 4-H4. Under this threshold spontaneous recovery from residual hematopoietic stem and progenitor cells may be expected within 30-50 days but going through cytopenic phases of granulocytic, megakaryocytic and erythrocytic lineages. HSCT should be considered if the victim's HSC pool is essentially irreversibly damaged. Interestingly, even after TBI, intrinsically radioresistant stem cells have been detected in distinct bone marrow (BM) areas comprising a residual hematopoietic stem and progenitor cell pool. However, these hematopoietic stem cells are not able to reconstitute hematopoiesis by themselves, if the TBI dose was myeloablative.

Acute radiation syndrome (ARS) does not only imply damage to the bone marrow. In a dose-dependent matter, it can also emerge as gastrointestinal and cerebrovascular syndromes leading to development of multiple organ dysfunctions. Damage to the whole organism is related to a systemic inflammatory response. Different target organs are affected due to activation of the innate immune system, resulting in a significant release of inflammatory cytokines. The pathophysiology appears comparable to that of acute graft-versus-host disease (GvHD) following allogeneic stem cell transplantation where a similar “cytokine storm” has been observed. Long-term effects of ionizing radiation have been well documented in atomic bomb survivors in whom persistent signs of inflammation, e.g. increased plasma levels of tumor necrosis factor-α (TNF-α), interferon-γ, interleukin-6, and C-reactive protein have been reported. Additionally, oxidative stress after high dose ionizing radiation has been involved in delayed morbidity.

The management of patients suffering from acute radiation syndrome is still a major challenge.

### SUMMARY OF THE INVENTION

Thus, it is an object of the present invention to provide a method of treating patients which have received a supralethal dose of total body irradiation which leads to the destruction of the hematopoietic stem cells to an extent that they cannot reconstitute endogenous hematopoiesis.

These and further objects of the invention, as will become apparent from the description, are attained by the subject-matter of the independent claims.

Some of the preferred embodiments of the present invention form the subject-matter of the dependent claims.

The present inventors have surprisingly found that mesenchymal stromal cells may be used to treat patients which have received such a high dose of total body irradiation that endogenous hematopoiesis cannot be restored.

Accordingly, in one embodiment, the present invention provides a method of treating a mammal which has received a myeloablative lethal dose of total body irradiation, comprising administering a therapeutically effective dose of mesenchymal stromal cells to the irradiated mammal.

In another embodiment, the present invention relates to a method of providing hematopoietic recovery in a mammal which has received a myeloablative lethal dose of total body irradiation, comprising administering a therapeutically effective dose of mesenchymal stromal cells to the irradiated mammal.

In still another embodiment, the present invention provides a method of increasing the survival of a mammal which has received a myeloablative lethal dose of total body irradiation, comprising administering a therapeutically effective dose of mesenchymal stromal cells to the irradiated mammal.

In a preferred embodiment of the present invention the mammal is selected from the group consisting of a rodent, a horse, a cow, a pig, a dog, a cat, a non-human primate and a human, more preferably the mammal is a human.

In a further preferred embodiment of the present invention the mesenchymal stromal cells have been cultured in a cell culture medium supplemented with platelet lysate.

More preferably, the mesenchymal stromal cells are obtained by a method comprising:
(a) providing bone marrow;
(b) culturing the bone marrow on tissue culture plates in culture medium for 2 to 10 days;
(c) removing non-adherent cells;
(d) culturing the adherent cells for 9 to 20 days in medium supplemented with platelet lysate; and
(e) removing the adherent cells from the tissue culture plates, thereby obtaining the mesenchymal stromal cells.

In a further embodiment of the present invention the mesenchymal stromal cells cultured in a medium supplemented with platelet lysate express at least one gene selected from the group consisting of heme oxygenase (HMOX1); potassium large conductance calcium-activated channel, subfamily M, beta member 1 (KCNMB1); crystallin, alpha B (CRYAB) and family with sequence similarity 5, member C (FAM5C) at a lower degree than mesenchymal stromal cells cultured in a medium supplemented with fetal calf serum.

In a further embodiment of the present invention the therapeutically effective dose of mesenchymal stromal cells is administered within 2 to 20 hours after the mammal has received the myeloablative lethal dose of total body irradiation.

### BRIEF DESCRIPTION OF THE DRAWINGS

FIG. 1: Mouse mesenchymal stromal cells (MSCs) rescue mice after total body irradiation.

Transplantation of bulk mMSCs led to a normalization of the peripheral white blood cell count within 4 weeks. Thrombocyte recovery needed approx. 8 weeks for normalization. Thus, results are comparable to blood recovery after HSC transplantation.

FIG. 2: Integration site pattern of clonal mMSCs. The clonal mMSCs were investigated using LM-PCR.

Each of the clones represents a specific pattern of integration sites. Bands marked with red asterisk were subjected to sequencing for further characterization. IC, internal control.

FIG. 3: Differentiation potential of mMSCs.

Mouse MSCs were generated from male C57BL/6J bone marrow and expanded for 9 passages. Expanded mMSCs were retrovirally transduced with eGFP (bulk) and cloned. Five clones with sufficient growth were selected and further expanded. They differed regarding their morphology and growth pace. Cells from passages 14-16 were induced to differentiate into adipogenic, osteogenic and chondrogenic cells. All clones and the parental bulk cells demonstrated three-lineage differentiation capability. Noninduced controls were negative for the respective stainings (not shown).

FIG. 4: Donor mMSCs are not detectable long-term.

(a) Tracking of eGFP-labeled clonal IXH8 donor mMSCs after transplantation revealed a fast decrease in PB. Within 8 hours, approx. 2% were quantified in PB and none after 10 days (n=8 for each time point). Insert: mMSCs accumulated in lungs (Lu) within 24 h and disappeared within 10 days (not shown). Spleen (Sp), liver (Li) and BM were negative at dl.

(b) Based on standard dilutions (filled symbols), no eGFP signals (open symbols) above the assay's detection limit of approx. 0.5% were detected in the BM of long-term survivors reconstituted with mMSCs of clone IXH8 (dashed line, detection limit of qPCR). nd, not detected.

FIG. 5: Spectral karyotyping of mMSCs.

(a) Shown is the SKY analysis of clone IXH8. SKY analysis revealed clonal structural and numerical chromosomal alterations as demonstrated in the spectral image of a representative diploid metaphase.

(b) In most metaphases, a hypertriploid (representative metaphase shown here) to an almost hypotetraploid chromosome complement with loss of the Y-chromosome was observed.

FIG. 6: Transplantation of mMSCs leads to gene expression changes in BM supporting rescue of endogenous hematopoiesis.

(a) Bootstrap hierarchical clustering of all 20K genes depicted highly stable clusters for HSC/BM vs. MSC.

(b) Heat map clustering using average distance and Manhattan metric for 2 microarrays per group hybridized with RNA from pooled BM (HSC: 2 animals/array, MSC: 3 animals/array) is shown for genes summarized in Table 3.

(c) Gene expression ratios of selected genes using BM of mice 21 days after transplantation with MSCs (n=9) or HSCs (n=10) were investigated for independent cohorts (grey columns: microarray data; white columns: quantitative PCR). Shown are mean ratios±SEM. P<0.005. Suggested functions of validated genes are shown in italics.

FIG. 7: Ectopic ossicles in mice lungs after i.v. transplantation of bulk mMSCs. Mice subjected to total body irradiation and transplanted i.v. with syngeneic MSCs were analyzed after 7 months. In lungs, fibrotic lesions were detected with HE staining (a) which showed the typical dark precipitates in von-Kossa stainings (b) admixed with large Collagen I-positive areas (c) suggesting bone and cartilage containing ossicles.

FIG. 8: Mechanism of reconstitution by MSCs: tracking of male donor cells in bone marrow of long-term survivors.

Based on standard dilutions of male with female DNA tested with qPCR (filled symbols), no male signals (open symbols) were detected in BM of mice transplanted with clonal IXH8 mMSCs. Y-chromosomal analysis in survivors proofed to be not reliable since clonal mMSCs after prolonged culture lost the Y-chromosome.

### DETAILED DESCRIPTION OF THE INVENTION

Before describing in detail exemplary embodiments of the present invention, the following general definitions are provided.

The present invention as illustratively described in the following may suitably be practiced in the absence of any element or elements, limitation or limitations, not specifically disclosed herein.

The present invention will be described with respect to particular embodiments but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. a cell is defined to be obtainable by a specific method, this is also to be understood to disclose a cell which is obtained by this method.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

The term “myeloablative lethal dose” is intended to mean an irradiation dose which destroys hematopoietic stem cells to an extent that endogenous hematopoiesis cannot be restored without the treatment of the present invention. A myeloablative lethal dose leads to a hematopoietic score of 4-H4 in the irradiated animals. The hematopoietic score may be determined by measuring the lymphocyte, granulocyte and/or platelet count after the animal has received the irradiation. Typically, in a human having a hematopoietic score of 4-H4 the lymphocytes decline within the first 24 hours and remain between 0.1 to 0.25×10⁹ cells/liter for several weeks. Further, granulocytes may increase within 48 hours, but then decline rapidly to values of ≦0.5×10⁹ cells/liter and remain at this value for several weeks. Finally, platelets decline over the first 10 days and remain low. Further information on the determination of the hematopoietic score may be found in Fliedner T M, Friesecke I, Beyrer K (2001) Medical Management of Radiation Accidents: Manual on the Acute Radiation Syndrome. Oxford: British Institute of Radiology and on http://www.remm.nlm.gov/ars.htm.

The term “endogenous hematopoiesis” is intended to mean the production of peripheral blood leukocytes derived from the animal's own hematopoietic stem cells.

The term “total body irradiation” (TBI) means the irradiation of the whole body and not only a part thereof with ionizing radiation such as X rays and gamma rays. The TBI may, for example, be the result of reactor accidents, the explosion of atomic bombs or nuclear terrorism.

The irradiation dose is usually measured in Gray, which is the SI unit of adsorbed radiation dose of ionizing radiation and is defined as the absorption of one joule of ionizing radiation by one kilogram of tissue (J/kg).

The person skilled in the art knows how to determine the “myeloablative lethal dose” for a given mammalian species. For example, if the dose is to be determined for mice, mice can be subjected to different doses of irradiation. The dose at which no animals survive four weeks after they have received the treatment with the irradiation is considered the myeloablative lethal dose. A typical myeloablative lethal dose for mice is between 9.5 and 11 Gy, preferably between 9.5 and 10.5 Gy, more preferably between 9.5 Gy and 10 Gy and most preferably it is 9.5 Gy. For humans, the myeloablative lethal dose is at least 8.5 or 9 Gy, preferably at least 9.5 or 10 Gy, more preferably at least 10.5, 11 or 11.5 Gy, even more preferably at least 12, 12.5 or 13 Gy and most preferably at least 15, 17 or 20 Gy.

Mesenchymal stromal cells (MSCs), which are also called marrow stromal cells or mesenchymal progenitor cells, are defined as self-renewable, multi-potent progenitor cells with a capacity to differentiate into several distinct mesenchymal lineages. MSCs have been demonstrated to differentiate into mesodermal-derived cells, e.g. bone, cartilage, fat and tendon. Their surface phenotype has been defined as being negative for CD45 and CD34 and positive for CD73 (SH4), CD105 (SH2) and CD90 (Thy-1). They can be isolated from the bone marrow by their ability to adhere to the plastic substrate of the cell culture plate which distinguishes them from hematopoietic cells which do not adhere to the plate. The minimal criteria for defining mesenchymal stromal cells are disclosed in Dominici et al. (2007) Cytotherapy 9(3): 301-302. Because most MSC populations lack specific cell surface markers, many isolation protocols are based on the process of negative selection meaning that e.g. bone marrow cells lacking the expression of endothelial and hematopoietic cell markers are sorted out and maintained as MSC cultures.

The differentiation of MSCs to the chondrogenic lineage can be initiated by resuspending the cells with 2% alginic acid and dropwise gelatinization in 0.1 M CaCl₂, whereas the osteogenic medium contains 100 nM dexamethasone, 10 mM β-glycerophosphate and 0.05 mM ascorbic acid (see Lange et al. (2005) Stem Cells and Development 14: 70-80). Supplements for differentiating the MSCs to the osteogenic or the adipogenic lineage are also commercially available from Stem Cell, Inc.

The induction of the chondrogenic differentiation can be detected by staining the MSC derived chondrogenic cells with 0.05% acidic Alcian Blue and 4% MgCl₂ after gelatinization for 10 minutes and 7 days of incubation with culture medium. The osteogenic differentiation can be detected by staining mineral deposits with 6% silver nitrate under UV light after two to six weeks of incubation in osteogenesis-inducing medium (see Lange et al. (2005) Stem Cells and Development 14: 70-80).

Preferably, the mesenchymal stromal cells used in the methods of the present invention have not been immortalized and/or have not been genetically manipulated. The term “immortalized” is intended to mean that the cells are not treated to prolong their normal lifespan and/or to prevent their apoptosis. The term “genetically manipulated” means that one or more genes are transferred into the cells by means of gene technology, e.g. by transfection or infection with suitable vectors.

The mesenchymal stromal cells are administered in a therapeutically effective dose, i.e. in a dose which is sufficient to achieve the desired effect of the treatment, e.g. providing hematopoietic recovery and/or increasing the survival in a mammal which has received a myeloablative dose of total body irradiation. The person skilled in the art knows how to determine the therapeutically effective dose of the MSCs for a given animal. An effective dose for treatment is determined by the body weight of the patient to be treated and the severity of the irradiation, i.e. the irradiation dose received.

In some embodiments of the methods of the present invention, from about 1×10⁵ to about 1×10⁸ MSCs per kilogram of recipient body weight are administered in a therapeutically effective dose. Preferably from about 5×10⁵ to about 5×10⁷ MSCs per kilogram of recipient body weight are administered in a therapeutic dose. More preferably from about 1×10⁶ to about 2×10⁷ MSCs per kilogram of recipient body weight are administered in a therapeutic dose. Most preferably about 2×10⁶ MSCs per kilogram of recipient body weight are administered in a therapeutic dose. The number of cells used will depend on the weight and condition of the recipient, the number of or frequency of administrations, and other variables known to those of skill in the art.

The MSCs may be administered once or several times. For example, if the MSCs are administered several times, they are administered 3-7 times over the course of 3-7 consecutive days.

The MSCs are administered within 2 to 20 hours, preferably within 3 to 15 hours, more preferably within 4 to 10 hours and most preferably within 4 to 8 hours after the mammal has received the myeloablative lethal dose of TBI.

The therapeutically effective dose of mesenchymal stromal cells is administered in a suitable solution for injection. Suitable solutions are those that are biologically and physiologically compatible with the cells and with the recipient, such as buffered saline solution, Plasma-lyte or other suitable excipients, known to one of skill in the art.

The mesenchymal stromal cells can be administered via any suitable administration route, preferably by intra-arterial or intravenous injection or infusion.

The present invention also relates to a method of providing hematopoietic recovery in a mammal which has received a myeloablative lethal dose of total body irradiation, comprising administering a therapeutically effective dose of mesenchymal stromal cells to the irradiated mammal. The term “providing hematopoietic recovery” is intended to mean that the level of blood cell populations such as leukocytes and thrombocytes returns to normal after treatment of the irradiated mammal with the mesenchymal stromal cells. In particular, a normal leukocyte level is obtained 3-5 weeks, preferably by 4 weeks after transplantation of the MSCs and a normal thrombocyte level is obtained by 6-10 weeks, preferably 7-9 weeks and more preferably 8 weeks after transplantation of the MSCs. A “normal” leukocyte or thrombocyte level means the level of leukocytes or thrombocytes, respectively, in an untreated, healthy animal. In mice, the normal leukocyte level is 4.5-8×10³ leukocytes per μl blood and the normal thrombocyte level is 550-1350×10³ thrombocytes per μl blood. In humans, the normal leukocyte level is 4.0-10×10³ leukocytes per μl blood and the normal thrombocyte level is 150-40350×10³ thrombocytes per μl blood.

To determine the level of blood cell populations, a blood sample may be obtained, e.g. from the retroorbital vein, and analyzed for the number of cells therein, for example using a hematology analyzer, e.g. COULTER® LH 500, COULTER® LH 750, COULTER® LH HmX, KX-21N hematology analyzer.

The present invention also relates to a method of increasing survival in a mammal which has received a myeloablative lethal dose of total body irradiation, comprising administering a therapeutically effective dose of mesenchymal stromal cells to the irradiated mammal. The term “increasing survival” is intended to mean an increase of the period of time that a mammal survives following exposure to a myeloablative lethal dose of total body irradiation by the method of the present invention. For example, without the treatment of the present invention mice die at the latest four weeks after they have received the irradiation. If treated according to the present invention at least 16, preferably at least 30, more preferably at least 40 and most preferably at least 50% of the mice survive for at least seven months and only some of the animals die at the earliest seven or eight weeks, preferably nine or ten weeks, more preferably eleven or twelve weeks and most preferably thirteen, fourteen or fifteen weeks after they have received the irradiation.

Alternatively or additionally, the term “increasing survival” means that in comparison to untreated mammals which have received the same myeloablative dose of total body irradiation, a higher percentage of mammals which have received the treatment of the present invention survives. For example, at least 16%, preferably at least 30% and more preferably at least 40% of the treated mammals survive for at least seven months after they have received the irradiation, whereas all of the untreated animals die within four weeks after they have received the myeloablative dose of total body irradiation.

In a preferred embodiment of the present invention the mesenchymal stromal cells are cultured in a cell culture medium supplemented with platelet lysate prior to transferring them to the irradiated mammal.

The term “cell culture” refers to the maintenance and propagation of cells and preferably animal (including human-derived) cells and most preferably mesenchymal stromal cells in vitro. Ideally the cultured cells do not differentiate and do not form organized tissues, but undergo mitosis synchronously.

“Cell culture medium” is used for the maintenance of cells in culture in vitro. For some cell types, the medium may also be sufficient to support the proliferation of the cells in culture. The medium provides nutrients such as energy sources, amino acids and anorganic ions and may also contain a dye like phenol red, sodium pyruvate, several vitamins, free fatty acids and trace elements.

The term “cell culture medium supplement” within the meaning of the present invention refers to a medium additive which is added to the medium to stimulate the proliferation of the cells. Usually this supplement will contain one or more growth factors which are responsible for the stimulation of proliferation. The term “supplement” is not intended to comprise medium additives which are added to the medium for the purpose of freezing the cells. In addition to the cell culture medium supplement, other compounds such as hormones, glutamine, ribonucleotides, desoxyribonucleotides and antibiotics, etc. may be added to the medium.

The term “maintenance of cells” is intended to mean that the cell number remains substantially unchanged, i.e. neither increases nor decreases.

The term “proliferation of cells” is intended to mean the multiplication of cells thereby leading to an increase in the cell number. The proliferation of cells may be detected by any suitable method. The easiest way to measure proliferation is to seed the cells in a specific, predetermined density and to count the cell number at different time points after seeding. Another way of measuring the proliferation of cells is a [³H]-thymidine incorporation assay which involves the addition of [³H]-thymidine to the cells, incubating them for a specific time, lysing the cells and counting the incorporation of [³H]-thymidine in a scintillation counter. Commercially available kits like the tetrazolium assay (MTT, Sigma) may also be used for measuring proliferation.

The term “growth factor” is intended to comprise proteins which stimulate proliferation of cells by binding to a specific receptor. Usually, growth factors only act on specific cell types which express the respective receptor for the growth factor. Examples of growth factors are epidermal growth factor (EGF), nerve growth factor (NGF), platelet derived growth factor (PDGF), fibroblast growth factor (FGF), vascular endothelial growth factor (VEGF), bone morphogenetic proteins (BMP), colony stimulating factors (CSF), transforming growth factor beta (TGF-β), insulin-like growth factor 1 (IGF-1), etc.

Platelets originate as cell fragments or “minicells” (without nuclear DNA) from megakaryocytes of the bone marrow. Platelets are characteristically activated at sites of injury where they create a physical barrier to limit blood loss and accelerate the generation of thrombin to intensify the coagulation process. Additionally, they are involved in wound healing and repair of mineralized tissue (Gentry (1992) Journal of Comparative Pathology 107: 243-270; Barnes et al (1999) Journal of Bone Mineral Research 14: 1805-1815). This latter function is mediated by the release of growth factors which are chemoattractants for mesenchymal cells of the external soft tissue and the bone marrow (Barnes et al (1999) Journal of Bone Mineral Research 14: 1805-1815).

The term “lysate” refers to the product of the lysis of cells, i.e. the product of disrupting the cellular integrity which leads to the release of the molecules which are normally present within the cells into the solution. The cellular integrity is disrupted by at least partially destroying the cell membrane.

The platelet lysate (PL) for use in culturing mesenchymal stromal cells is prepared from a solution that has a platelet concentration of 1×10⁸ to 1×10¹⁰ per ml, preferably of 5×10⁸ to 5×10⁹ per ml and most preferably of approximately 1-3×10⁹ per ml.

The concentration of the supplement in the cell culture medium is between 1-20% v/v, preferably between 2-18% v/v, more preferably between 3-12% v/v and most preferably approximately 5% v/v.

As the supplement comprising the platelet lysate provides growth factors which are necessary for the growth of cells and which are released from the platelets upon lysis of the cells, the concentration of the supplement in the medium and the platelet concentration from which the supplement is prepared are linked with each other. Therefore, a lower concentration of supplement in the medium can be used, if the lysate is prepared from a solution with a high platelet concentration and accordingly, a higher concentration of a supplement is necessary, if the lysate is prepared from a solution with a low platelet concentration.

In particular, if the lysate is prepared from a platelet solution with a concentration of 1.5-3×10⁹ per ml, a supplement concentration in the medium of 5% is used.

The skilled person knows methods how to determine the optimal combination of the concentration of the platelets in the solution from which the lysate is prepared and the concentration of the supplement in the medium. For example, one can prepare a lysate from solutions with different concentrations of platelets and add these lysates in different concentrations to the medium. Afterwards, the proliferation rate of the cells is compared and the combination is chosen which gives the highest proliferation rate. The proliferation rate may be determined by seeding a defined number of cells which is the same for each condition, counting the cells at different time points after seeding and comparing the growth rate from the different conditions. Such proliferation experiments are within the routine work of the skilled artisan.

The type of medium which is supplemented with the cell culture medium supplement of the present invention depends on the type of cells which is to be cultured in the cell culture medium. The skilled person knows how to select the medium which is suitable for culturing a particular cell type. For culturing MSCs, for example, alpha-MEM, DMEM and DMEM/F12 are suitable. Further suitable media are IMDM, Optimem, DMEM/LG/L-G, DMEM/HG/L-G, DMEM/HG/GL, DMEM/LG/GL, alpha-MEM/L-G, alpha-MEM/GL (Life Technologies; Sotiropoulou et al. (2006) Stem Cells 24(5): 1409-1410) and Poietics Human Mesenchymal stromal cell Medium (M2; PT-3001, Cambrex; Wagner et al. (2005) Exp. Hem. 33(11): 1402-1416).

Preferably, the medium for culturing the MSCs is alpha-MEM. This medium is characterized by the presence of lipoic acid, sodium pyruvate, ascorbic acid and vitamin B12. The medium may be purchased from companies such as Cambrex, Invitrogen, Sigma-Aldrich and Stem Cell Technologies.

In a preferred embodiment, the starting material for the MSCs is bone marrow isolated from healthy donors. Preferably, these donors are mammals. More preferably, these mammals are humans, if the MSCs are to be used for treating humans. In one embodiment, the bone marrow is cultured on tissue culture flasks between 2 and 10 days, preferably 2 to 3 days prior to washing non-adherent cells from the flask. Preferably the bone marrow is cultured in platelet lysate (PL) containing media. For example, 300 μl of bone marrow is cultured in 15 ml of PL supplemented medium in T75 or other adequate tissue culture dishes.

After washing away the non-adherent cells, the adherent cells are also cultured in media that has been supplemented with platelet lysate (PL). In one embodiment of the method of producing MSCs, an optimized preparation of PL is used. This optimized preparation of PL is made up of pooled platelet rich plasmas (PRPs) from at least 10 donors (to equalize for differences in cytokine concentrations) with a minimal concentration of 3×10⁹ thrombocytes/ml.

The term “platelet-rich plasma (PRP)” refers to a concentration of platelets in a carrier which concentration is above that of platelets normally found in blood. For example, the platelet concentration may be 2 times, 5 times, 10 times, 100 times or more of the normal concentration in blood.

PRP may comprise blood components other than platelets. It may be 50% or more, 75% or more, 80% or more, 95% or more, 99% or more platelets. The non-platelet components may be plasma, white blood cells and/or any blood component. PRP may be obtained using autologous, allogeneic, or pool sources of platelets and/or plasma.

PRP may be prepared in different ways which are summarized in Zimmermann et al. (2001) Transfusion 41: 1217-1224, including apheresis, the tube method and the preparation from buffy coat units or pooled thrombocyte concentrates.

Additionally, there are different commercially available systems for the preparation of PRP like Vivostat PRF Preparation Kit®, PCCS Platelet Concentrate Collection System®, Harvest® SmartPReP 2 APC 60 Process and Fibrinet® Autologous Fibrin and Platelet System. These systems are compared in Leitner et al. (2006) Vox Sanguinis 91: 135-139.

According to preferred embodiments of the invention, PL is prepared either from pooled thrombocyte concentrates designed for human use or from 7-13 pooled buffy coats after centrifugation with 200×g for 20 min. Preferably, the PRP is aliquoted into small portions, frozen at −80° C., and thawed immediately before use. PL-containing medium is prepared freshly for each cell feeding. In a preferred embodiment, medium contained αMEM as basic medium supplemented with 5 IU Heparin/ml medium (source: Ratiopharm) and 5% of freshly thawed PL.

In one embodiment of the invention, the adherent cells are cultured in PL-supplemented media at 37° C. with approximately 5% CO₂ under hypoxic conditions. Preferably, the hypoxic conditions are an atmosphere of 5% O₂. In some situations hypoxic culture conditions allow MSCs to grow more quickly. This allows for a reduction of days needed to grow the cells to 90-95% confluence at which the cells are detached from the culture plate. Generally, it reduces the growing time by three days.

In another embodiment, the adherent cells are cultured in PL-supplemented media at 37° C. with approximately 5% CO₂ under normoxic conditions, i.e. wherein the O₂ concentration is the same as atmospheric O₂, i.e. approximately 20.9%.

Preferably, the adherent cells are cultured between 9 and 20 days, preferably for 10 to 15 days, being fed every 4 days with PL-supplemented media. The expert knows that the cultivation time depends on the density of the cells when they are seeded. If the cells are seeded in a high density, they will be cultured for a shorter time than cells which are seeded in a low density. Alternatively or additionally, the cells are passaged one to six times, preferably 3 to 4 times.

In one embodiment, the adherent cells are grown to between 90 and 95% confluence. Preferably, once this level of confluence is reached, the cells are trypsinized to release them from the plate.

In certain embodiments, the population of cells that is isolated from the plate is between 85-95% MSCs, as determined by flow cytometry analysis. In other embodiments, the MSCs are greater than 95% of the isolated cell population.

In another embodiment of the invention, the cells are frozen after they are released from the tissue culture plate. Freezing is performed in a step-wise manner in a physiologically acceptable carrier, 20% serum albumin and 10% DMSO. Preferably, the serum albumin is human serum albumin.

Thawing is also performed in a step-wise manner. Preferably, when thawed, the frozen MSCs are diluted to remove DMSO. If the MSCs are to be administered intra-arterially, the DMSO has to be diluted from the cells. If the MSCs are to be administered intravenously, then it is not important to dilute the DMSO from the cells. In this case, frozen MSCs of the invention are thawed quickly at 37° C. and administered intravenously without any dilution or washings.

Optionally the cells are administered following any protocol that is adequate for the transplantation of hematopoietic stromal cells (HSCs), for example the protocol described in Stockschläder et al. (1995) Bone Marrow Transplant. 1995 15(4): 569-572.

In another embodiment, the cells are frozen in aliquots of 1-5×10⁸ cells in 50 mL of physiologically acceptable carrier and serum albumin (HSA). In one aspect of this embodiment, when a therapeutic dose is being assembled, the appropriate number of cells is thawed for the therapeutic dose. Preferably, after DMSO is diluted from the thawed cells, the cells are placed in a sterile infusion bag with 5% serum albumin. Preferably the serum albumin is human serum albumin. Preferably the albumin is present at a concentration of 5% w/v. Once in the bag, the MSCs do not aggregate and viability remains greater than 95% even when the MSCs are stored at room temperature for at least 6 hours. This provides ample time to administer the MSCs to a patient. Suspending the 10⁶-10⁸ cells in greater than 40 mL of physiological carrier is critical to their biological activity. If the cells are suspended in lower volumes, the cells are prone to aggregation. Administration of aggregated MSCs to mammalian subjects has resulted in pulmonary infarction. Thus, it is crucial that non-aggregated MSCs be administered according to the methods of the invention. The presence of albumin is also critical because it prevents aggregation of the MSCs and also prevents the cells from sticking to plastic containers the cells pass through when administered to subjects. Optionally, the physiologically acceptable carrier is Plasma-lyte.

In another embodiment, a closed system is used for generating and expanding the MSCs from bone marrow of normal donors. This closed system is a device to expand cells ex vivo in a functionally closed system. In one specific embodiment, the closed system includes: 1. a central expansion unit preferably constructed similarly to bioreactors with compressed (within a small unit), but extended growth surfaces; 2. media bags which can be sterilely connected to the expansion unit (e.g. by welding tubes between the unit and the bags) for cell feeding; and 3. electronic devices to operate automatically the medium exchange, gas supply and temperature.

The advantages of the closed system in comparison to conventional flask tissue culture are the construction of a functionally closed system, i.e. the cell input and media bags are sterile welded to the system. This minimizes the risk of contamination with external pathogens and therefore may be highly suitable for clinical applications. Furthermore, this system can be constructed in a compressed form with consistently smaller cell culture volumes but preserved growth area. The smaller volumes allow the cells to interact more directly with each other which creates a culture environment that is more comparable to the in vivo situation of the bone marrow niche. Also the closed system saves costs for the media and the whole expansion process.

The construction of the closed system may involve two sides: the cells are grown inside of multiple fibres with a small medium volume. In some embodiments, the culture media contains growth factors for growth stimulation, and medium without expensive supplements is passed outside the fibres. The fibres are designed to contain nanopores for a constant removal of potentially growth-inhibiting metabolites while important growth-promoting factors are retained in the growth compartment.

In certain embodiments, the closed system is used in conjunction with a medium for expansion of MSCs which does not contain any animal proteins, e.g. fetal calf serum (FCS). FCS has been associated with adverse effects after in vivo application of FCS-expanded cells, e.g. formation of anti-FCS antibodies, anaphylactic or arthus-like immune reactions or arrhythmias after cellular cardioplasty. FCS may introduce unwanted animal xenogeneic antigens, viral, prion and zoonose contaminations into cell preparations making new alternatives necessary.

It has been found that MSCs cultured in a medium containing platelet lysate are less immunogenic than MSCs cultured in a medium containing FCS as shown in vitro in mixed lymphocyte cultures (MLC). In mixed lymphocyte cultures, peripheral blood lymphocytes from two individuals are mixed in tissue culture for several days and the stimulation of the lymphocytes is measured by suitable means, e.g. thymidine uptake. Optionally, the lymphocytes from one individual may be inactivated by radiation or treatment with mitomycin so that the activation of only one lymphocyte population is measured.

MSCs have been described to act immunomodulatory by impairing T-cell activation without inducing anergy. A dilution of this effect has been shown in mixed lymphocyte cultures (MLC) if decreasing amounts of MSCs are added to the MLC reaction, leading eventually to an activation of T-cells. This activation process is not observed when PL-generated MSCs are used in the MLC as third party. Thus, MSCs are less immunogenic after PL-expansion and FCS seems to act as a strong antigen or at least has adjuvant function in T-cell stimulation.

Further, when the expression of certain genes in MSCs cultured in a medium supplemented with platelet lysate is compared with the expression of these genes in MSCs cultured in a medium supplemented with FCS, a differential expression of genes involved in inflammation and/or apoptosis can be observed. For example, at least one, preferably at least two, more preferably at least three and most preferably all four genes selected from the group consisting of heme oxygenase (HMOX1); potassium large conductance calcium-activated channel, subfamily M, beta member 1 (KCNMB1); crystallin, alpha B (CRYAB) and family with sequence similarity 5, member C (FAM5C) are expressed at a lower degree in mesenchymal stromal cells cultured in a medium supplemented with platelet lysate than in mesenchymal stromal cells cultured in a medium supplemented with fetal calf serum.

In particular, the expression of HMOX1 is decreased by a factor of 9 to 12, preferably 10 to 11, in the MSCs cultured in platelet lysate, the expression of KCNMB1 is decreased by a factor of 8 to 11, preferably 9 to 10, in the MSCs cultured in platelet lysate, the expression of CRYAB is decreased by a factor of 7 to 10, preferably 8 to 9, in the MSCs cultured in platelet lysate and the expression of FAM5C is decreased by a factor of 6 to 9, preferably 7 to 8, in the MSCs cultured in platelet lysate.

The skilled person knows how to determine the expression of genes in the cells. For example, RNA may be isolated from the cells and analyzed by RT-PCR using suitable primers, by Northern Blot using suitable probes or by a suitable microarray. Primers or probes for the above-mentioned genes can be developed on the basis of the publicly available sequence information. The nucleotide sequence of human HMOX1 is available on the NCBI homepage under the accession number NM_{—}002133, the nucleotide sequence of human KCNMB_{—}1 is available on the NCBI homepage under the accession number NM_{—}004137, the nucleotide sequence of human CRYAB is available on the NCBI homepage under the accession number NM_{—}001885 and the nucleotide sequence of human FAM5C is available on the NCBI homepage under the accession number NM_{—}199051.

While lower dose radiation victims may profit from supportive care, the situation is more serious after irradiation dose higher than 6 Gy. The only curative treatment in these cases is the transplantation of HSCs. However, this alternative depends on whether a suitable donor is available and can be provided quickly. The normal time required to find and deliver a HSC transplant spans over weeks, a frame too long for seriously affected individuals.

HSCs reside in close association with osteoblasts and sinusoidal blood vessels within the bone marrow and this association contributes to the maintenance of the HSC pool in vivo. Self-renewal, proliferation and differentiation of HSCs are regulated through intrinsic signals from the BM niche in which the MSCs are a regulatory component.

It has now been found that lethally irradiated mice fully reconstitute the blood system through transplantation of mMSCs with similar kinetics as HSCs. Systemic administration of non-clonal mMSCs result in long-term survival of the majority of animals with normal blood cell distribution.

Generation and expansion of MSCs from C57BL/6J mice is particularly difficult and time-consuming. Since it is well known that rodent adherent BM cells might contain HSCs over long periods caused by emperiopoiesis among other mechanisms, we formally cannot exclude a contamination of MSC preparations with remaining HSCs. Therefore, clonal mMSCs were evaluated for their reconstitution potential. Here, surprisingly, one population of cloned mMSCs showed an even better reconstituting potential than the bulk population. This clone IXH8 was different from all other MSC cultures: a) morphologically, the clone consisted exclusively of spindle shaped cells indicative for significantly accelerated proliferation of MSCs; that was not observed in bulk and other clonal cultures, and b) flow-cytometrically, we detected increased hematopoietic CD34 and CD45 but no CD105 expression. CD105 has been described as a marker of proliferation and adhesion.

Lack of CD105 may increase survival of recipients by lowering lung embolization. All other characteristics corresponded to the ISCT criteria, questioning the relevance of CD105 expression particularly for mMSC characterization. Likewise, overexpression of CD105 in cultured endothelial cells has been shown to induce a marked increase in protein levels of inflammatory eNOS, suggesting an anti-inflammatory action of CD105-negative mMSCs in our model. Surprisingly, none of the recipients transplanted with clonal mMSCs developed osteosarcomas or fibrotic lesions in lungs as has been observed with non-clonal cultures (FIG. 7).

Taking together the differences in morphology, antigen expression and survival, we conclude that the composition of the expanded mMSCs (e.g. significant amount of nonproliferating osteoprogenitors) rather than the source of the population (mMSC or hMSC) causes different outcomes after transplantation.

The survival of recipient animals suggests the homing of mMSCs to the BM. We tested this using either the Y-chromosome of male donor cells or the stably integrated eGFP as detection marker. Y-chromosome-based chimerism analysis in female recipients could not detect donor cells in any investigated tissue including PB and BM, although animals survived long-term. Spectral karyotyping of clonal mMSCs revealed various structural chromosomal alterations and massive aneuploidy (as did bulk mMSCs) with loss of the Y-chromosome whereas bulk cultures of passage13 were still Y-positive. Hence, mMSCs not only accumulate chromosomal abnormalities during few in vitro passages but also might lose sex-specific chromosomes. Despite this, no tumors or osseous inclusions were formed as shown in FIG. 7. We assume that cloning of mMSCs selected defined populations which do not contain replicative senescent cells as has been regularly detected in bulk populations. The clonal mMSCs may not be prone to stable lung embolization and thus do not lead to eventual tumorous degeneration.

Quantitative PCR for stably integrated eGFP-sequences also failed to detect any donor cells and no eGFP-positive cells were found in PB, BM or thymus by flowcytometry. These results were unexpected, since forced in vitro differentiation of human MSCs suggested a potential differentiation capability into hematopoietic and endothelial cells, albeit to a rather low degree.

Although it cannot be completely ruled out the presence of single donor cells in the investigated tissues below the detection limit, hematopoietic recovery in recipients due to replenishment with donor cells is unlikely in the present setting. Additionally, transplantation of cells with the shown high incidence of chromosomal aberrations would in high probability result in tumor formation. That was not the case in the recipients pointing at limited survival of the cells in vivo. This conclusion contradicts the results of earlier studies analyzing hematopoietic recovery after myeloablative TBI with blood-derived mMSCs and showing donor characteristics in blood and BM (Lange et al. (1999) J. Hemother. Stem Cell Res. 8: 335-342; Huss et al. (2000) Stem Cells 18: 252-260). One fundamental difference between both cell sources is the immortalization of cells with SV40 used in these studies, potentially altering BM homing capability of MSCs.

In contrast to MSCs described here, mesodermally derived multipotent adult progenitor cells (MAPCs) with their extraordinarily high plasticity are unable to radioprotect lethally irradiated recipients but possess long-term multilineage hematopoietic repopulating activity, thus preceding HSCs in ontogeny. However, their in vivo equivalent and the true nature (e.g. in vitro artifact) are still unknown. MAPCs seem to differ fundamentally from MSCs in their in vitro and in vivo differentiation potential.

Kinetic analysis of the distribution of eGFP+ donor cells after i.v. transplantation substantiated the fast disappearance from PB. Mouse MSCs trapped in lungs quickly, however without long-term residence and embolization as revealed by lack of donor signals 10 days post-transplant. Additionally, no homing of donor mMSCs to the BM was evident, pointing to salvage of endogenous irradiation-surviving HSCs but not to reconstitution of hematopoiesis by donor MSCs. This conclusion is corroborated by the gene expression profile in BM of MSC-transplanted animals. MSCs in a complex mechanism counteracted factors, e.g. oxidative stress, inflammation and toxification by degradation products which suppress the recovery of remaining HSCs. The MSC-mediated regulation of the niche environment likely is a redundant system that is mediated by several molecules as has been shown for immunoregulation by MSCs.

Irradiation produces excessive inflammatory responses, contributing to HSC death if left untreated. Among other organs, the lung is especially sensitive towards irradiation damage and may retain MSCs. MSCs interfere with inflammation by changing the gene expression profile not only in the lungs where they dock but also in BM. To do so, MSCs need not necessarily home to the BM but might globally change gene expression or activate the production of systemically counteracting substances. This mechanism has been described for MSCs influencing myocardial infarction.

Our results provide evidence for a highly effective trophic mechanism working also in BM after lethal irradiation in mice. MSCs may also be used in alleviating myelosuppression due to chemotherapy and toxic drug reaction. Because BM-derived MSCs are easily accessible, can be massively expanded, and stored for prolonged time, they can easily be distributed to places in need. Hence, MSC-infusion is provided as an efficient and immediate treatment option after irradiation injuries.

Human bone marrow mesenchymal stromal cells (hMSCs) are currently investigated for a variety of therapeutic applications. However, most expansion protocols still use fetal calf serum of (FCS), a growth factor supplement which is a potential source of undesired xenogenic pathogens.

As previously described, compared to fetal calf serum supplemented culture conditions, we found a significant increase in both colony forming unit-fibroblast (CFU-F) as well as cumulative cell numbers after expansion in MSCs cultured in a medium supplemented with platelet lysate. The accelerated growth may be optimized by pooling of at least 10 thrombocyte concentrates. It may be suitable to use 5% of PL with an optimal platelet concentration of 1.5-3×10⁹/ml and centrifugation of platelet lysate at high speed.

Cells expanded by procedures described herein meet all criteria for mesenchymal stromal cells, e.g. plastic adherence, spindel shape morphology, surface marker expression, lack of hematopoietic markers and differentiation capability into 3 mesenchymal lineages. MSCs of the passage 6 were cytogenetically normal, and characterized by the immune privileged potential to suppress allogenic reaction of T-cells. Additionally, gene expression profiles showed increased RNA levels of genes involved in cell cycle and DNA replication and downregulation of developmental and differentiation genes supporting the observation of increased MSC expansion in platelet-supplemented medium. Growth kinetics of PL-MSCs are favourable compared to fetal calf serum expanded MSCs leading to 3 log higher yield day 80 post initiation of culture.

PL-MSCs differ significantly from fetal calf serum expanded MSCs. Immunosuppression of mixed lymphocyte cultures can be observed even at very diluted doses of MSCs.

Fetal calf serum cultured cells will lead to stimulation of T-cells at lower doses. This observed immunosuppression is one effect of the below described radiation rescue. The radiation damage is not only caused by direct damage to the cell, but also by inflammatory reaction of the environment to the stem cells. These inflammatory reactions can better be suppressed by PL-MSCs than by FCS-MSCs.

In one embodiment, mouse mesenchymal stromal cells (mMSCs) were expanded from bone marrow, retrovirally labelled with eGFP (bulk cultures) and cloned. Bulk and five selected clonal mMSCs populations were characterized in vitro for their multilineage differentiation potential and phenotype showing no contamination with hematopoietic cells. Lethally irradiated recipients were i.v. transplanted with bulk or clonal mMSCs. We found a long-term survival of recipients with fast hematopoietic recovery after the transplantation of MSCs exclusively without support by HSCs. Quantitative PCR based chimerism analysis detected eGFP-positive donor cells in peripheral blood immediately after injection and in lungs within 24 hours. However, no donor cells in any investigated tissue remained long-term. Despite the rapidly disappearing donor cells, microarray and quantitative RT-PCR gene expression analysis in the bone marrow of MSC-transplanted animals displayed enhanced regenerative features characterized by (i) decreased proinflammatory, ECM formation and adhesion properties and (ii) boosted anti-inflammation, detoxification, cell cycle and anti-oxidative stress control as compared to HSC-transplanted animals.

The data presented in the Examples section reveal that systemically administered MSCs provoke a protective mechanism counteracting the inflammatory events and also supporting detoxification and stress management after radiation exposure. Further, these results indicate that MSCs, their release of trophic factors and their HSC-niche modulating activity rescue endogenous hematopoiesis thereby serving as fast and effective first-line treatment to combat radiation-induced hematopoietic failure.

Other gene functions regulated by PL expansion are described in the following table:

| Gene | Fold expr. in | | |
|---|---|---|---|
| description | PL-MSCs | Proposed function | reference |
| leucine-rich | 15.4 | Decreased bone | Kim T, Kim K, Lee S H, So H S, Lee J, Kim N, Choi |
| repeat (LRR) | | formation | Y. Identification of LRRc17 as a negative regulator of |
| protein | | | receptor activator of NF-kappaB ligand (RANKL)- |
| (P37NB) | | | induced osteoclast differentiation. J Biol Chem. 2009 |
| | | | May 29; 284(22): 15308-16 |
| claudin 11 | 12.1 | tight junctions, role | Lal-Nag M, Morin P J. The claudins. Genome Biol. |
| (CLDN11) | | in tissue integrity | 2009; 10(8): 235 |
| RAR-responsive | 9.7 | inhibition of MSC | Ohnishi S, Okabe K, Obata H, Otani K, Ishikane S, |
| (TIG1) | | differentiation into | Ogino H, Kitamura S, Nagaya N. Involvement of |
| | | osteocytes and the | tazarotene-induced gene 1 in proliferation and |
| | | expression of | differentiation of human adipose tissue-derived |
| | | osteocalcin | mesenchymal stem cells. Cell Prolif. 2009 |
| | | | June; 42(3): 309-16. |
| keratin 18 | 8.4 | role in filament/cell | Ku N O, Omary M B. Identification of the major |
| (KRT18) | | formation/cell | physiologic phosphorylation site of human keratin 18: |
| | | stability | potential kinases and a role in filament |
| | | | reorganization. J Cell Biol. 1994 October; 127(1): 161-71. |
| breast cancer | 8.8 | maintenance of | Tutt A, Ashworth A. The relationship between the |
| 2, early onset | | genome stability | roles of BRCA genes in DNA repair and cancer |
| (BRCA2) | | | predisposition. Trends Mol Med. 2002 December; 8(12): 571- |
| | | | 6. |
| insulin-like | −23.2 | involved in | Chao W, D'Amore P A. IGF2: epigenetic regulation |
| growth factor | | development and | and role in development and disease. Cytokine |
| 2 (IGF2) | | growth | Growth Factor Rev. 2008 April; 19(2): 111-20. |
| glycoprotein | −13.9 | membrane | Fjorback A W, Müller H K, Wiborg O. Membrane |
| M6B | | trafficking and cell- | glycoprotein M6B interacts with the human serotonin |
| (GPM6B | | to-cell | transporter. J Mol Neurosci. 2009 March; 37(3): 191-200. |
| | | communication | |
| ring finger | −11.5 | methylating/silencing | Kim J, Guermah M, McGinty R K, Lee J S, Tang Z, |
| protein 20 | | function during | Milne T A, Shilatifard A, Muir T W, Roeder R G. |
| (RNF20) | | transcription | RAD6-Mediated transcription-coupled H2B |
| | | | ubiquitylation directly stimulates H3K4 methylation |
| | | | in human cells. Cell. 2009 May 1; 137(3): 459-71. |
| heme | −10.6 | proangiogenic | Dulak J, Deshane J, Jozkowicz A, Agarwal A. Heme |
| oxygenase | | antiinflammatory, | oxygenase-1 and carbon monoxide in vascular |
| (decycling) | | antioxidant, and | pathobiology: focus on angiogenesis. Circulation. |
| 1(HMOX1) | | antiapoptotic | 2008 Jan. 15; 117(2): 231-41. |
| | | effects | |
| potassium | −9.7 | apoptosis | (Xie M J, Ma Y G, Gao F, Bai Y G, Cheng J H, Chang |
| large | | | Y M, Yu Z B, Ma J. Activation of BKCa channel is |
| conductance | | | associated with increased apoptosis of |
| calcium- | | | cerebrovascular smooth muscle cells in simulated |
| activated | | | microgravity rats. Am J Physiol Cell Physiol. 2010 |
| channel, | | | June; 298(6): C1489-500. |
| subfamily M, | | | |
| beta member | | | |
| 1 | | | |
| (KCNMB1) | | | |
| MHC class | −9.4 | | increased immunogenicity/T cell activation |
| II, DR alpha | | | |
| (HLA-DRA) | | | |
| dermatan | −8.6 | interacting with | |
| sulfate | | collagen fibrils and | |
| proteoglycan | | other extracellular | |
| 3 (DSPG3) | | matrix proteins | |
| crystallin, | −8.6 | modification of | Watanabe G, Kato S, Nakata H, Ishida T, Ohuchi N, |
| alpha B | | p53-dependent | Ishioka C. alphaB-crystallin: a novel p53-target gene |
| (CRYAB) | | apoptosis | required for p53-dependent apoptosis. Cancer Sci. |
| | | | 2009 December; 100(12): 2368-75. |
| family with | −7.9 | correlated to | Carvalho F M, Tinoco E M, Deeley K, Duarte P M, |
| sequence | | inflammatory/ | Faveri M, Marques M R, Mendonça A C, Wang X, |
| similarity 5, | | immunological factors | Cuenco K, Menezes R, Garlet G P, Vieira A R. |
| member C | | | FAM5C contributes to aggressive periodontitis. PLoS |
| (FAM5C) | | | One. 2010 Apr. 7; 5(4): e10053. |

Without being bound to this theory, we conclude that MSCs grown in PL-supplemented media are more protective against ischemia reperfusion damage and radiation injury than MSCs grown in fetal calf serum supplemented medium.

Further exemplary embodiments of the present invention include:

Described herein is a method which may comprise administering PL-MSC from an allogenic but otherwise identical donor mammal to an irradiated mammal, thereby rescuing the mammal from a lethal dose of total body irradiation.

In one embodiment, the mammal is selected from the group consisting of a rodent, a horse, a cow, a pig, a dog, a cat, a non-human primate, and a human. In another embodiment, the mammal is a human.

In another embodiment, the administration is infusion.

Also described herein is a method of enhancing hematopoiesis in a mammal. The method may comprise administering PL-MSC from an allogenic but otherwise identical donor mammal to a mammal, thereby enhancing hematopoiesis in the mammal.

In one embodiment, the mammal is selected from the group consisting of a rodent, a horse, a cow, a pig, a dog, a cat, a non-human primate, and a human. In another embodiment, the mammal is a human.

In another embodiment, the administration is infusion.

In addition, there is provided a method of enhancing hematopoietic stem cell differentiation in a mammal given a lethal dose of total body irradiation. The method may comprise administering PL-MSC from an allogenic but otherwise identical donor mammal to an irradiated mammal, thereby enhancing hematopoietic stem cell differentiation in the mammal.

In one embodiment, the mammal is selected from the group consisting of a rodent, a horse, a cow, a pig, a dog, a cat, a non-human primate, and a human. In another embodiment, the mammal is a human.

In another embodiment, the administration is infusion.

Also provided is a method of enhancing the hematopoietic recovery in a mammal given a lethal dose of total body irradiation. The method may comprise administering PL-MSC from an allogenic but otherwise identical donor mammal to an irradiated mammal, thereby enhancing the hematopoietic recovery in said mammal.

A method of treating a mammal comprising an ablated marrow is also described herein. The method may comprise administering PL-MSC from an allogenic but otherwise identical donor mammal to a mammal, thereby treating the mammal comprising an ablated marrow.

Further, a method of enhancing hematopoiesis in a mammal comprising an ablated marrow is provided. The method comprises administering PL-MSCs from an allogenic but otherwise identical donor mammal to a mammal, thereby enhancing hematopoiesis in the mammal comprising an ablated marrow.

Further, a method of increasing the survival of a mammal exposed to a lethal dose of total body irradiation is provided. The method may comprise administering marrow stromal cells from an allogenic but otherwise identical donor mammal to an irradiated mammal, thereby increasing the survival of a mammal exposed to a lethal dose of total body irradiation.

According to one embodiment, mesenchymal stromal cells are provided that are isolated from bone marrow. Further, methods of producing these mesenchymal stromal cells are provided. The bone marrow may be cultured on tissue culture plates for 2-10 days. After this period, non-adherent cells can be removed and the remaining adherent cells can be cultured for an additional 9-20 days in platelet lysate (PL)-supplemented media. When the cells reach 80-90% confluence, the cells can be removed from the tissue culture plates. These cells may comprise from 85 to 95% mesenchymal stromal cells (MSCs). The cells can be suspended in a physiologically acceptable solution with approximately 20% serum albumin and 10% DMSO and frozen at a rate of 1° C. per minute temperature decrease.

In some embodiments, mesenchymal stromal cells are provided that have been cultered in platelet lysate supplemented culture media (PL-MSC). In this embodiment, the population of mesenchymal stromal cells may express Pickle 1 at a higher degree than mesenchymal stromal cells that have been cultured in fetal calf serum supplemented culture media.

In some embodiments, the mesenchymal stromal cells obtained by methods described herein, e.g. PL-MSC, are less immunogenic than mesenchymal stromal cell that have been cultured in fetal calf serum supplemented culture media.

In another embodiment, mesenchymal stromal cells, e.g. PL-MSC, are provided that express antigens selected from the group consisting of CD105, CD90, CD73 and MHC I on their surfaces. In some embodiments, mesenchymal stromal cells, e.g. PL-MSC, are provided that do not express proteins selected from the group consisting of CD45, CD34 and CD14 on their surface.

The invention is further described by the following examples, which are solely for the purpose of illustrating specific embodiments of this invention, and are not to be construed as limiting the scope of the invention in any way.

### EXAMPLES

Transplantation of female CD45.1 mice with male CD45.2 mMSCs were carried out the following way. Recipient mice were conditioned with 9.5 Gy total body irradiation (TBI) and subsequently transplanted with 10⁶ mMSCs/animal (bulk GFP+). A total of 28 mice were treated with MSCs, 15 animals served as irradiation control. The MSC-transplanted mice survived long-term whereas the radiation control did not survive day 21 (Table 1).

### Materials and Methods

### Mouse MSC Generation and Characterization.

Female C57BL/6J-CD45.1 mice (The Jackson Laboratory) represented the recipient population; male C57BL/6J mice with the wild-type CD45 (CD45.2) were used as donor animals.

Mouse MSCs were isolated from male bone marrow and expanded for 9 passages in DMEM/Ham's F12 medium (Biochrom) supplemented with 20% preselected fetal calf serum and 2 mM glutamine (both: Invitrogen). The cells were retrovirally labeled with SF91-eGFP at MOI=3 (bulk population) and expanded or seeded for cloning into ten 96-well plates at 0.3 cells/well. Expanded bulk and selected clonal mMSCs of P14-P18 were characterized according to their differentiation capability into adipo-, chondro- and osteogenic lineages and phenotype as described according to ISCT criteria (Dominici et al. (2007) Cytotherapy 9(3): 301-302).

Characterization of integration site pattern of clonal mMSCs was carried out as described via LM-PCR (Cornils et al. (2009) Mol. Ther. 17: 131-143). Genomic DNA was digested with Tsp509I giving rise to integration specific fragments of unique lengths. Linker cassettes with known sequences were ligated to the restriction sites. Primers were designed to bind at the known sequences of the provirus and the linker cassette. Amplified PCR products were loaded onto the gel. After gel extraction the bands (red stars) were sequenced. Using the BLAST algorithm, the obtained sequences were aligned to the mouse genome to identify the integration sites.

Chromosome preparation and spectral karyotyping of eGFP-transduced bulk-mMSCs, passage 13 and clone IXH8, passage 20 were performed as described previously (Rudolph and Schlegelberger (2009) Methods Mol. Biol. 506: 453-466).

### Mouse MSC Transplantation.

TBI was performed using a Cs-137 radiation source. Lethally (9.5 Gy) irradiated female C57BL/6J-CD45.1 mice were i.v. transplanted within 8 hours with 10⁶ cells divided into following groups: (i) male bulk (n=28) or cloned (n=59; irradiation controls n=15) mMSCs of P15-P20 for investigation of long-term survival; (ii) clone IXH8 mMSCs (n=32) to investigate the in vivo distribution of donor mMSCs; (iii) BM (n=4, named HSC) or bulk mMSCs (n=6, named MSC) for microarray analysis; (iv) HSC (n=10) or bulk mMSCs (n=9) to validate the differential gene expressions obtained with the microarray.

### Sample Acquisition and Examination.

From experimental animals of group (i) blood samples were taken retroorbitally and cell counts analyzed using a Coulter Onyx. Seven months later, peripheral blood, bone marrow, thymus, lymph node, liver, spleen, lung, intestine, aorta/vena cava and abdominal fat were removed and used for genomic detection of the Y-chromosome (Sry) and/or eGFP via quantitative PCR. Parts of lung tissues were fixed in formalin, paraffin embedded and serial cuts stained with HE, von-Kossa stain visualizing calcium precipitates and Collagen I.

Genomic DNA from PB and BM was isolated the same day using innuPREP Blood DNA Mini kit (Analytik Jena, Jena, Germany), DNA from all other snap frozen organs was isolated using the Invisorb Spin Tissue Mini Kit (Invitek, Berlin, Germany). To yield a standard curve, male/GFP-positive DNA was diluted at decreasing concentrations in female/GFP-negative DNA. Quantitative PCR reactions were performed on an Mx3000P (Stratagene). The reaction mixture contained 50 ng of DNA, SYBR Premix Ex Taq (Takara Bio INC), and 200 nM primers (MWG-BIOTECH AG). The threshold cycle (Ct) was determined for each reaction by the MxPro Software. Amplification efficiency was calculated by the sliding window method (LinReg software). Normalization of expression values was done using Rps27a for eGFP and control chro11 for Y-chromosome tests. Additionally, parts of freshly isolated PB, BM and thymus were investigated for donor derived eGFP- and CD45.2-antigen-expression using flow cytometry as described (Cornils et al. (2009) Mol. Ther. 17: 131-143).

Blood samples from animals of group (ii) were taken at different time points (20 min, 2, 4, 8, 12 and 24 hours; n=8 for each time point) after transplantation and isolated DNA was used for Sry/eGFP-chromosomal quantitative PCR. At day 1 and 10, PB, BM, lung, liver and spleen were obtained for DNA isolation. All samples were quantitatively tested for a) the Y-chromosome of recipient cells and b) the stably integrated eGFP.

From experimental animals of group (iii) at d21, BM from HSC- and mMSC-transplanted groups was flushed combining the cells of 2 or 3 mice respectively and used for RNA isolation using Invisorb Spin Cell RNA Mini Kit (Invitek). Non-manipulated BM of age-matched mice (n=4, named BM) was used as control. Differential gene expression was investigated using CodeLink UniSet Mouse 20K I Bioarrays as described previously (Lange et al. (2007) J. Cell Physiol. 213: 18-26). For each group (BM, HSC, MSC), two arrays were hybridized. Gene expression profiles of all genes were grouped by hierarchical clustering (TIGR MeV v.4.5.1; Manhattan distance, average linkage). All data is MIAME compliant and the raw and processed data has been deposited in a MIAME compliant database at the gene expression omnibus (GEO) under accession GSE21867.

Validation of differential gene expression of genes with a fold change of 2.5 was done on the independent mouse group (iv) using d21 BM of 10 animals transplanted with HSCs and 9 animals transplanted with clonal IXH8 mMSCs. Primers used are listed in Table S2. Differences in gene expressions in BM of MSC and HSC transplanted animals were determined based on the Ct method normalized for Taf12 and Rps27a.

### Statistical Analysis.

For statistical analysis, unpaired and two-tailed Student's t-test was applied. P-values<0.05 were considered statistically significant.

### Results

MSCs Promote Hematopoietic Recovery after Lethal Irradiation

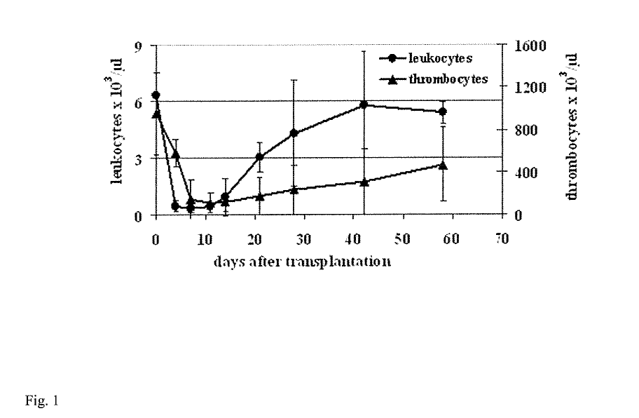

Dynamic evaluation of peripheral blood counts of animals treated with bulk MSCs revealed similar leukocyte and thrombocyte recovery as observed in recipients transplanted with HSCs reaching normalization of white blood cells after 4 weeks (FIG. 1). Seven months after transplantation, ⅔ of recipients still were alive (Table 1) and hematologically well with a normal distribution of peripheral blood cell (PB) populations (Table 2).

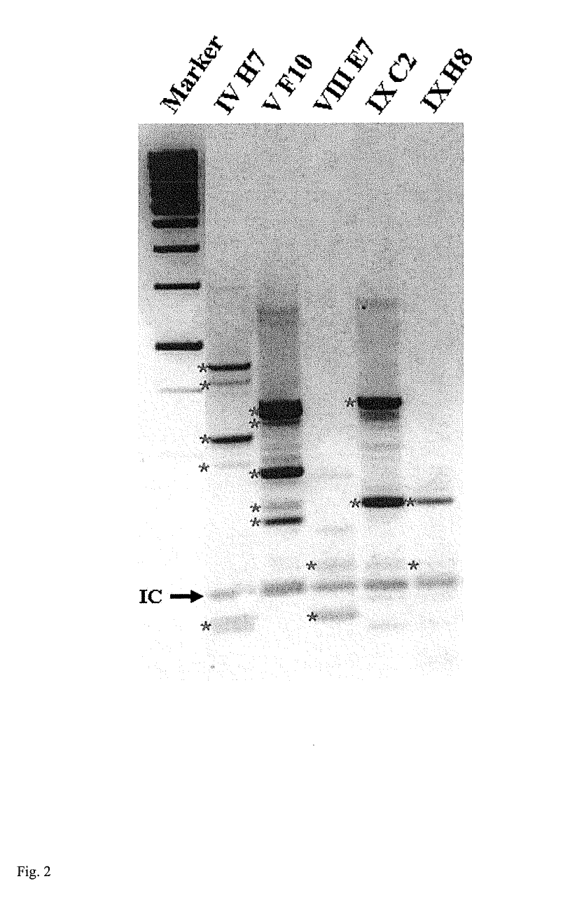

Using ligation-mediated (LM-) PCR specific integration site (IS) patterns for each single clone were identified in vitro (FIG. 2). For mMSC clone IVH7 2 IS, for VF10 3 IS, for VIIIE7 1 IS, for IXC2 2 IS and for IXH8 HIS were identified. Integration sites shown twice might be due to incomplete digestion of the genomic DNA. The thickness of the bands does not resemble minor or major integration sites but is inherent to the method.

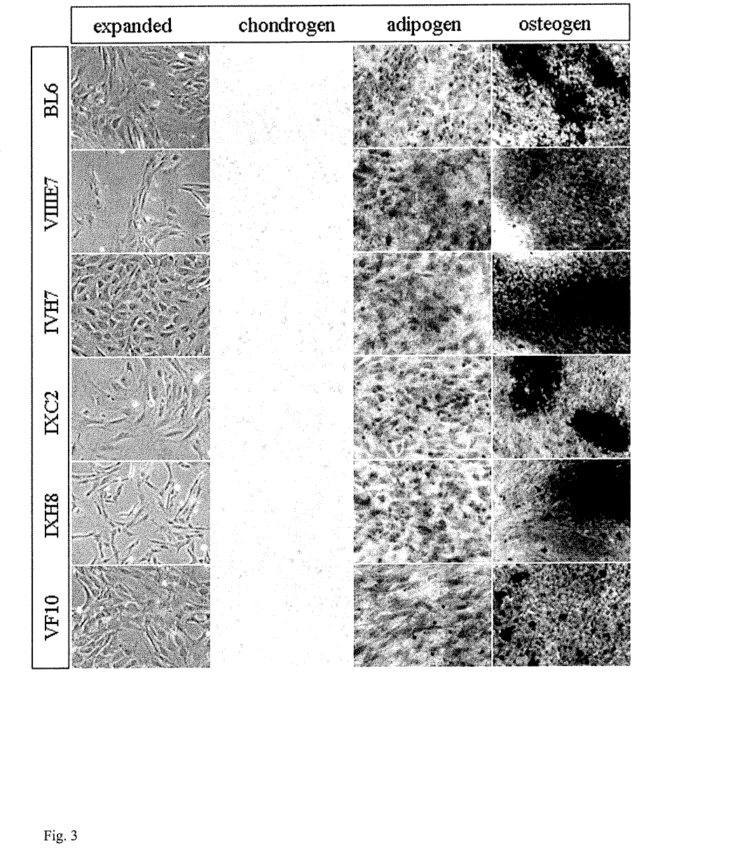

Transplantation of clonal mMSCs resulted in a superior survival rate of recipients treated with clone IXH8 (88%) compared to survival rates of approx. 30% obtained with other clones (Table 1). No control animals without cell transplantation survived the TBI longer than 3 weeks Impressively, clone IXH8 morphologically was different from all other cultures (FIG. 3) without any flattened cells and additionally showed a distinct phenotype with increased CD34 and CD45 but no CD105 expression. All other characteristics of this clone corresponded to the ISCT criteria (FIG. 3, Table 1), questioning the relevance of CD105 expression for MSC characterization.

### Transplanted Donor Cells are Detectable Short- but not Long-Term

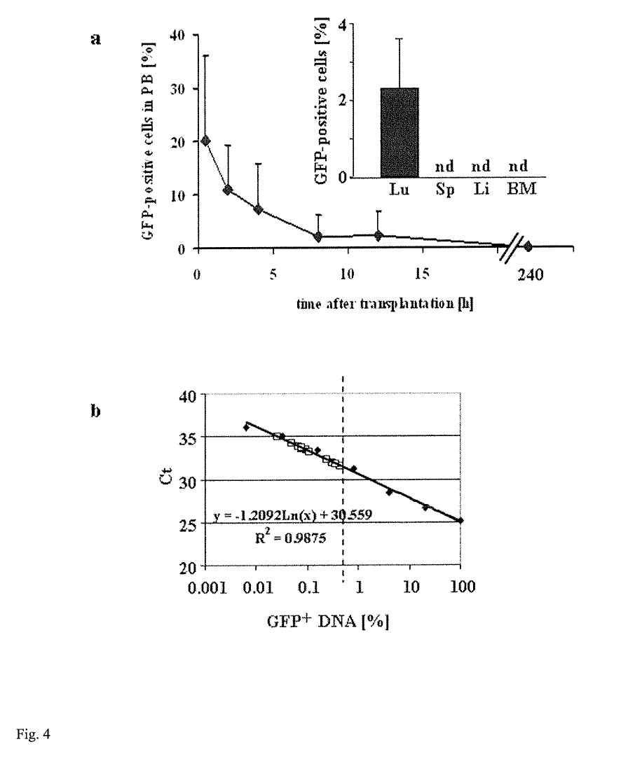

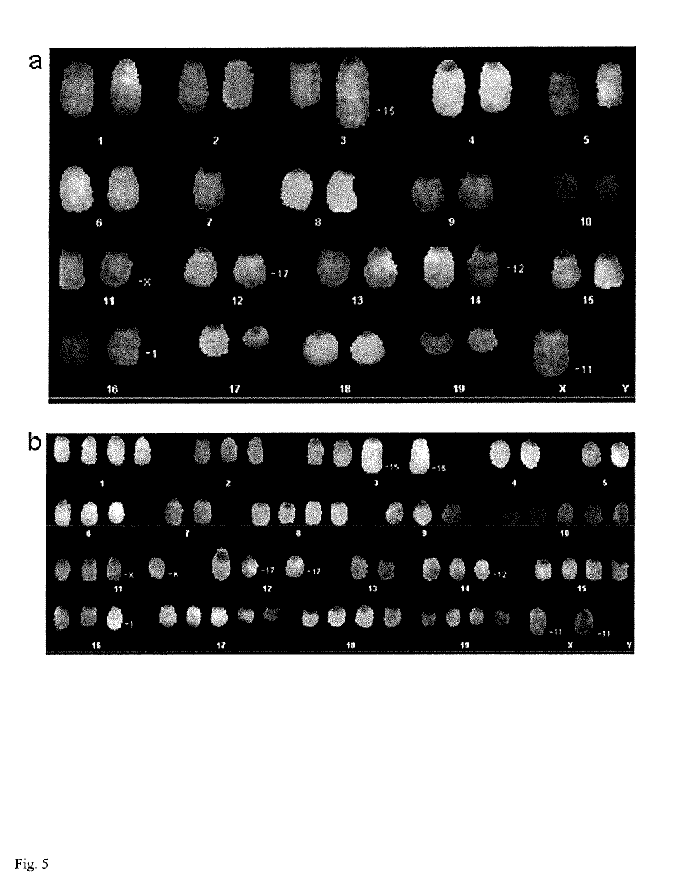

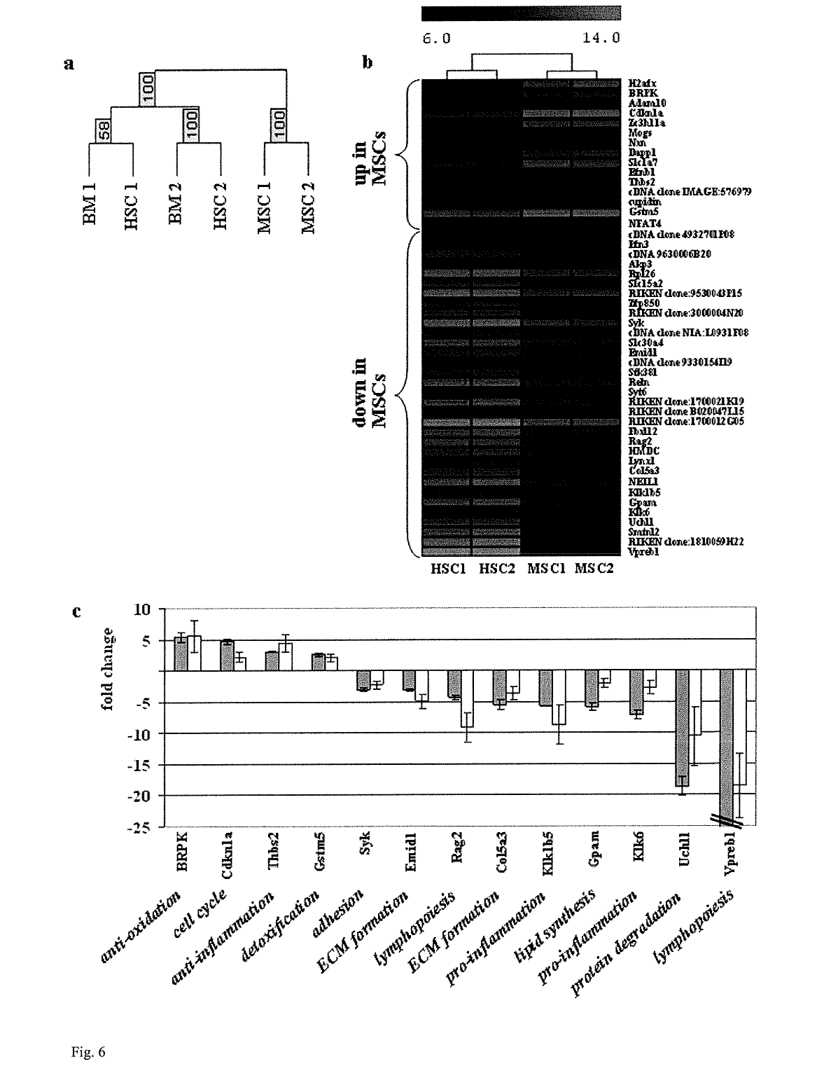

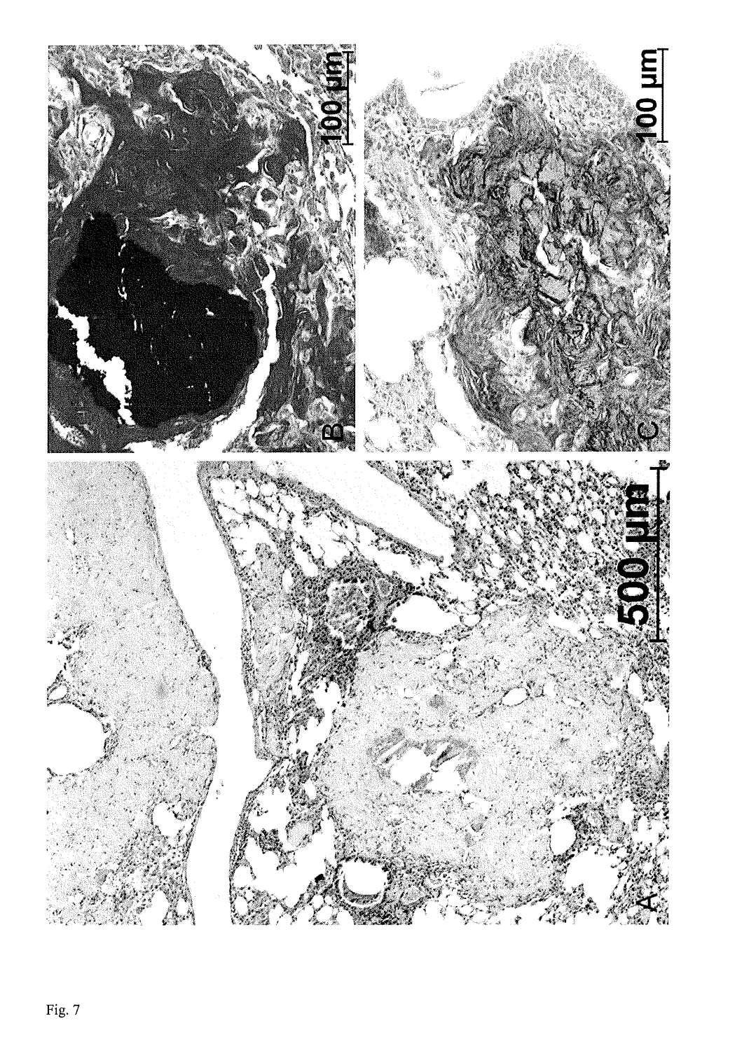

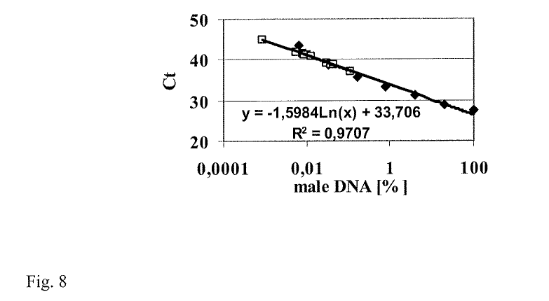

Stably integrated eGFP-sequences were used for tracing donor cells in recipients after transplantation. Immediately after injection, 20.2%±15.7 of transfused cells could be detected in the peripheral blood (FIG. 4a). Within 24 hours, eGFP-positive donor cells were diluted out from PB and trapped in lungs but not in BM, spleen or liver (FIG. 4a insert). At day 10 and later on, no donor cells remained in any investigated tissue (PB, thymus, lymph node, liver, spleen, lung, intestine, aorta/vena cava and abdominal fat; not shown). A standard curve for eGFP-BM and assessment of donor cells in long-term survivors (see Materials and Methods) is shown in FIG. 4b. This corresponds to results from repeated PB flow cytometry analysis of recipients within the 7-month period where no eGFP-positive or CD45.2 donor cells were found (not shown). Although male donor mMSCs were transplanted into female recipients, the Y-chromosome was not available for chimerism analysis. Among various structural chromosomal alterations and massive aneuploidy the loss of the Y-chromosome in clonal mMSCs was surprisingly detected already during the in vitro expansion period using spectral karyotyping (FIG. 5).

### MSCs Salvage Endogeneous Hematopoiesis

While donor mMSCs did not home to the BM, the gene expression profile in BM changed significantly, clustering as a separate group compared to HSC transplanted mice or age-matched BM (FIG. 6a). Impressively, the clustering of all genes was done without any preselection giving rise to highly stable clusters. A heat map using hierarchical clustering of up- and downregulated genes in the MSC compared to the HSC groups with p≦0.01 and fold changes of ≧2.5 or ≦2.5 illustrates the variations between the samples (FIG. 6b). Successful validation of selected genes by quantitative PCR (Table 3) emphasized the beneficial role of mMSCs in endogenous hematopoietic reconstitution (FIG. 6c). Transplantation of mMSCs upregulated genes in the BM involved in cell cycle and protection from oxidative stress (Cdkn1a, BRPK) as well as in anti-inflammatory and detoxification processes (Thbs2, Gstm5). In contrast, genes for enhancing inflammation (Klk6, Klk1b5), protein degradation (Uch11), adhesion/matrix formation (Sykb, Emid1, Co15a3), lipid synthesis (Gpam), and lymphoid development (Vpreb1, Rag2) were downregulated. The downregulation of genes involved in adhesion and matrix formation particularly suggests lower retention of hematopoietic progenitor cells within the stroma and higher potency to egress into the peripheral circulation.

Production of Mesenchymal Stromal Cells in Media Supplemented with Platelet Lysate

300 μl whole bone marrow was plated in 15 ml of αMEM media containing 5% PL in a tissue culture flask with 75 cm² of growth area or in larger vessels for 6-10 days to allow the mesenchymal stromal cells (MSCs) to adhere. Residual non-adherent cells were washed from the flask. αMEM media containing 5% platelet-rich plasma was added to the flask. Cells were allowed to grow until 70% confluency (approximately 3-4 days). Cells were then trypsinized and re-plated into a Nunc Cell Factory™. Cells remained in the Cell Factory™ for approximately 10-15 days for expansion with media exchanges every 4 days.

Cells were harvested by first washing in phosphate buffered saline (PBS), treating with trypsin and washing with αMEM and then cryopreserved in 10% DMSO, 5% human serum albumin and Plasmalyte using controlled-rate freezing. When the cells were required for infusion, they were thawed, washed free of DMSO and resuspended to the desired concentration in Plasmalyte containing 5% human serum albumin.

The final cell product consisted of approximately 10⁵-10⁸ cells per kg of weight of the subject (depending on the dose schedule) suspended in 50 ml Plasmalyte with 5% Human serum albumin. No growth factors, antibodies, stimulants, or any other substances were added to the product at any time during manufacturing. The final concentration was adjusted to provide the required dose such that the volume of product that is returned to the patient remained constant.

### Tables

TABLE 1
Phenotypical characterization of mMSCs and recipients' survival rates
after transplantation.
Cultures of eGFP-transduced bulk and cloned mMSCs after extended expansion
were positive for CD59, CD105 and Sca-1 but negative for the hematopoietic
markers CD34, CD45, CD117 and for CD90 by flow cytometry. Clone IXH8 was
different from all other cultures in its expression of CD34/CD45 and negativity of
CD105 (shown in bold italic). Transplantation with this clone resulted in the highest
survival rate of the irradiated recipients, suggesting elevated CD34 and CD45 and no
CD105 expressions might be a prerequisite of the high rescue capability. nd, not
done.

| | | | | | | | | survival at 7 |
|---|---|---|---|---|---|---|---|---|
| | CD34 | CD45 | CD59 | CD90 | CD105 | CD117 | Sca-1 | months [%] |
| bulk | 1.6 | 0.5 | 95.4 | 0.5 | 85.9 | 0.9 | 96.7 | 19/28 [67.9] |
| IXH8 | [symbol] | [symbol] | 97.4 | 2.7 | [symbol] | 1.5 | 99.2 | 15/17 [88.2] |
| IVH7 | 1.2 | 1.3 | 54.7 | 0.5 | 94.1 | 2.8 | 81.9 | 2/12 [16.7] |
| IXC2 | 0.9 | 2.2 | 79.6 | 1.2 | 94.0 | 1.5 | 90.2 | 3/10 [30] |
| VIIIE7 | 1.2 | 1.1 | 71.1 | 2.0 | 93.1 | 1.5 | 96.4 | 4/10 [40] |
| VF10 | 2.2 | 2.2 | 45.9 | 0.7 | 74.0 | 3.4 | 77.9 | 3/10 [30] |
| radiation control | nd | nd | nd | nd | nd | nd | nd | 0/15 [0] |

TABLE 2
Peripheral blood cell populations in mMSC transplanted animals.
The distribution of white blood cells 5 months after bulk mMSC
transplantation was counted using Pappenheim-stained blood smears.

| lymphocytes | neutrophils | monocytes | eosinophils |
|---|---|---|---|
| 72% ± 3 | 21% ± 3 | 5% ± 2 | 2% ± 1 |

TABLE 3
Differential gene expression in bone marrow of mice transplanted with
HSCs or MSCs.
Mean gene expression levels from microarray analysis in bone marrow at d21 after
transplantation were calculated for 2 arrays per group hybridized with RNA from
pooled BM (HSC: 2 animals/array, MSC: 3 animals/array). Selected were genes with
p ≦ 0.01 after t-test filtering and ratio of ≧2.5 or ≦2.5. References for suggested
functions were selected in the context of cell functionality after transplantation and
are shown in supplementary text.

| | | | | | | |
|---|---|---|---|---|---|---|
| Accession | | Mean | Mean | | | Suggested |
| No | Gene Name | HSC | MSC | ratio | p-value | functions |

Upregulated in MSCs

| | | | | | | |
|---|---|---|---|---|---|---|
| NM_010436 | H2A histone family, member | 7.18 | 11.05 | 14.65 | 0.0076 | DNA repair |
| | X (H2afx) | | | | | |
| AF316872 | protein kinase BRPK/PINK1 | 7.52 | 9.94 | 5.36 | 0.0082 | Protection from |
| | (Pten induced putative kinase | | | | | oxydative stress |
| | 1) | | | | | |
| NM_007399 | a disintegrin and | 5.69 | 7.94 | 4.74 | 0.0097 | shedding of |
| | metalloprotease domain 10 | | | | | inflammation |
| | (Adam10) | | | | | driving |
| | | | | | | substrates |
| NM_007669 | cyclin-dependent kinase | 10.19 | 12.42 | 4.68 | 0.0031 | Cell cycle |
| | inhibitor 1A (P21) (Cdkn1a) | | | | | control |
| NM_144530 | zinc finger CCCH type | 9.03 | 11.09 | 4.18 | 0.0016 | Signal |
| | containing 11A (Zc3h11a) | | | | | transduction |
| NM_020619 | glucosidase 1 (Gcs1), | 6.29 | 8.29 | 4.00 | 0.0067 | Reentry in the |
| | mannosyl-oligosaccharide | | | | | mitotic cell |
| | glucosidase (Mogs) | | | | | cycle, actin |
| | | | | | | cyto-skeletal |
| | | | | | | organization |
| NM_008750 | nucleoredoxin (Nxn) | 6.14 | 7.93 | 3.46 | 0.0079 | accelerated |
| | | | | | | proliferation |
| | | | | | | after oxidative |
| | | | | | | stress |
| NM_011932 | dual adaptor for | 8.73 | 10.40 | 3.18 | 0.0055 | lymphocyte |
| | phosphotyrosine and 3- | | | | | proliferation |
| | phosphoinositides 1 (Dapp1), | | | | | |
| | synonym for BAM32 | | | | | |
| NM_009201 | solute carrier family 1, | 9.33 | 11.00 | 3.18 | 0.0079 | Na+-dependent |
| | member 7 (Slc1a7) | | | | | amino acid |
| | | | | | | transporter |
| NM_010110 | ephrin B1 (Efnb1) | 5.15 | 6.82 | 3.18 | 0.0063 | angiogenic |
| | | | | | | remodelling |
| NM_011581 | thrombospondin 2 (Thbs2) | 6.02 | 7.65 | 3.10 | 0.0020 | antiinflammatory |
| AA116457 | mp95d10.r1 | 5.63 | 7.14 | 2.85 | 0.0089 | unknown |
| | Soares_thymus_2NbMT | | | | | |
| | cDNA clone IMAGE: 576979 | | | | | |
| | 5′ | | | | | |
| AB017136 | cupidin/HOMER 2, complete | 6.36 | 7.83 | 2.77 | 0.0029 | regulation of T- |
| | cds | | | | | cell cytokine |
| | | | | | | production |
| NM_010360 | glutathione S-transferase, mu | 11.15 | 12.54 | 2.62 | 0.0096 | detoxification |
| | 5 (Gstm5) | | | | | |
| D85612 | NFATx (NFAT4), complete | 7.90 | 9.27 | 2.57 | 0.0046 | Ca²⁺ dependent |
| | cds | | | | | T-cell activation |

Downregulated in MSCs

| | | | | | | |
|---|---|---|---|---|---|---|
| AV277818 | RIKEN full-length enriched, | 7.83 | 6.50 | 2.52 | 0.0094 | unknown |
| | adult male testis (DH5a) | | | | | |
| | cDNA clone 4932701P08 3′, | | | | | |
| NM_029303 | profilin 3 (Pfn3), mRNA | 6.67 | 5.30 | 2.58 | 0.0051 | actin filament |
| | | | | | | assembly (cell |
| | | | | | | motility) |
| NM_172555 | RIKEN cDNA 9630006B20 | 10.09 | 8.71 | 2.60 | 0.0018 | Ion channel |
| | gene poly(A) polymerase | | | | | |
| | gamma (Papolg) | | | | | |
| NM_007432 | alkaline phosphatase 3, | 6.15 | 4.74 | 2.65 | 0.0051 | fat absorption |
| | intestine, not Mn requiring | | | | | |
| | (Akp3) | | | | | |
| NM_009080 | ribosomal protein L26 (Rpl26) | 12.03 | 10.60 | 2.70 | 0.0030 | irradiation- |
| | | | | | | induced |
| | | | | | | apoptosis |
| NM_021301 | solute carrier family 15 | 10.39 | 8.95 | 2.70 | 0.0068 | peptide transport |
| | (H+/peptide transporter), | | | | | |
| | member 2 (Slc15a2) | | | | | |
| AK020595 | adult male urinary bladder | 11.94 | 10.49 | 2.73 | 0.0098 | RNase |
| | cDNA, RIKEN | | | | | |
| | clone: 9530043P15 | | | | | |
| | product: hypothetical | | | | | |
| | Pancreatic ribonuclease | | | | | |
| BM240956 | K0609H05-3 NIA | 9.67 | 8.17 | 2.83 | 0.0064 | unknown |
| | Hematopoietic Stem Cell (Lin-/ | | | | | |
| | c-Kit-/Sca-1+) cDNA clone | | | | | |
| | NIA: K060, Zfp850 (zinc | | | | | |
| | finger protein 850) | | | | | |
| AK013872 | 12 days embryo head cDNA, | 10.03 | 8.47 | 2.95 | 0.0004 | unknown |
| | RIKEN clone: 3000004N20 | | | | | |
| | product: hypothetical RNA- | | | | | |
| | binding region RNP-1 | | | | | |
| NM_011518 | spleen tyrosine kinase (Syk) | 11.86 | 10.29 | 2.96 | 0.0059 | adhesion |
| | | | | | | regulation |
| BM119878 | L0931F08-3 NIA Newborn | 9.11 | 7.52 | 3.01 | 0.0083 | unknown |
| | Kidney cDNA Library (Long) | | | | | |
| | cDNA clone NIA: L0931F08 | | | | | |
| | IMAGE: 30003043 3′ | | | | | |
| NM_011774 | solute carrier family 30 (zinc | 10.708 | 9.08 | 3.06 | 0.0052 | enzyme activity |
| | transporter), member 4 | | | | | |
| | (Slc30a4) | | | | | |
| NM_080595 | EMI domain containing 1 | 9.71 | 8.09 | 3.08 | 0.0034 | ECM formation |
| | (Emid1) | | | | | |
| BB078651 | RIKEN full-length enriched, | 6.99 | 5.365 | 3.10 | 0.0066 | unknown |
| | adult male diencephalon | | | | | |
| | cDNA clone 9330154I19 3′ | | | | | |
| NM_172734 | serine/threonine kinase 38 like | 9.33 | 7.61 | 3.30 | 0.0090 | neural |
| | (Stk381) | | | | | differentiation |
| NM_011261 | reelin (Reln) | 11.03 | 9.25 | 3.44 | 0.0041 | neural |
| | | | | | | differentiation |
| NM_018800 | synaptotagmin 6 (Syt6) | 7.18 | 5.39 | 3.47 | 0.0021 | vesicular transport |
| | | | | | | proteins |
| AK006217 | adult male testis cDNA, | 11.07 | 9.19 | 3.70 | 0.0099 | unknown |
| | RIKEN clone: 1700021K19 | | | | | |
| | product: hypothetical Serine- | | | | | |
| | rich region/Cysteine-rich | | | | | |
| BB713538 | RIKEN full-length enriched, 2 | 8.82 | 6.90 | 3.79 | 0.0041 | unknown |
| | cells egg cDNA clone | | | | | |
| | B020047L15 3′, mRNA | | | | | |
| | sequence | | | | | |
| AK005904 | adult male testis cDNA, | 12.91 | 10.95 | 3.88 | 0.0044 | unknown |
| | RIKEN clone: 1700012G05 | | | | | |
| | product: RIKEN cDNA | | | | | |
| | 1700012G05 gene | | | | | |
| NM_013911 | F-box and leucine-rich repeat | 11.07 | 9.03 | 4.11 | 0.0098 | ubiquitin- |
| | protein 12 (Fbxl12) | | | | | mediated |
| | | | | | | proteolysis |
| NM_009020 | recombination activating gene | 10.72 | 8.61 | 4.31 | 0.0032 | lymphoid |
| | 2 (Rag2) | | | | | development |
| BC037381 | 3-hydroxymethyl-3- | 10.15 | 7.98 | 4.48 | 0.0095 | phospholipid |
| | methylglutaryl-Coenzyme A | | | | | biosynthesis |
| | lyase-like 1 (HMDC) | | | | | |
| NM_011838 | Ly6/neurotoxin 1 (Lynx1) | 8.81 | 6.48 | 5.01 | 0.0091 | cell development |
| NM_016919 | collagen, type V, alpha 3 | 10.12 | 7.69 | 5.39 | 0.0072 | Structure of |
| | (Col5a3), mRNA | | | | | fibrillar collagen |
| AK013322 | 10, 11 days embryo whole | 11.33 | 8.86 | 5.53 | 0.0041 | DNA repair |
| | body cDNA, RIKEN clone: | | | | | after oxydative |
| | 2810450N13 | | | | | damage |
| | product: hypothetical | | | | | |
| | Formamidopyrimidine-DNA | | | | | |
| | glycolase containing protein/ | | | | | |
| | NEIL1 | | | | | |
| NM_008456 | kallikrein 1-related peptidase | 8.95 | 6.47 | 5.58 | 0.00006 | ECM |
| | b5 (Klk1b5) | | | | | proteolysis, |
| | | | | | | pro- |
| | | | | | | inflammation |
| NM_008149 | glycerol-3-phosphate | 10.94 | 8.40 | 5.80 | 0.0035 | glycerolipid |
| | acyltransferase, mitochondrial | | | | | synthesis |
| | (Gpam) | | | | | |
| NM_010639.5 | kallikrein 6 (Klk6) | 8.67 | 5.84 | 7.13 | 0.0029 | ECM |
| | | | | | | proteolysis, |
| | | | | | | pro- |
| | | | | | | inflammation |
| NM_011670 | ubiquitin carboxy-terminal | 10.15 | 5.94 | 18.52 | 0.0008 | Protein |
| | hydrolase L1 (Uchl1) | | | | | degradation |
| BC037993 | smoothelin-like 2 (Smtnl2) | 11.12 | 6.41 | 26.10 | 0.0031 | Actin stress |
| | | | | | | fibers, |
| | | | | | | inhibition of |
| | | | | | | vasodilation |
| AK007907 | 10 day old male pancreas | 12.27 | 6.66 | 49.02 | 0.0024 | unknown |
| | cDNA, RIKEN | | | | | |
| | clone: 1810059H22 | | | | | |
| | product: hypothetical protein | | | | | |
| NM_016982 | pre-B lymphocyte gene 1 | 14.41 | 8.25 | 71.59 | 0.0030 | B cell |
| | (Vpreb1) | | | | | development |

1. A method of treating a mammal which has received a myeloablative lethal dose of total body irradiation, comprising administering a therapeutically effective dose of mesenchymal stromal cells to the irradiated mammal.

2. The method of claim 1, wherein the therapeutically effective dose of mesenchymal stromal cells is effective to provide hematopoietic recovery in the mammal.

3. The method of claim 1, wherein the therapeutically effective dose of mesenchymal stromal cells is effective to increase the survival of the mammal.

4. The method of claim 1, wherein the mesenchymal stromal cells are isolated from bone marrow.

5. The method of claim 1, wherein the mammal is selected from the group consisting of a rodent, a horse, a cow, a pig, a dog, a cat, a non-human primate and a human.

6. The method of claim 5, wherein the mammal is a human.

7. The method of claim 1, wherein the mesenchymal stromal cells have been cultured in a cell culture medium supplemented with platelet lysate.

8. The method of claim 7, wherein the mesenchymal stromal cells are obtained by a method comprising:
a. providing bone marrow;
b. culturing the bone marrow on tissue culture plates in culture medium for 2 to 10 days;
c. removing non-adherent cells;
d. culturing the adherent cells for 9 to 20 days in medium supplemented with platelet lysate; and
e. removing the adherent cells from the tissue culture plates, thereby obtaining the mesenchymal stromal cells.

9. The method of claim 7, wherein the mesenchymal stromal cells cultured in a medium supplemented with platelet lysate express at least one gene selected from the group consisting of heme oxygenase (HMOX1); potassium large conductance calcium-activated channel, subfamily M, beta member 1 (KCNMB 1); crystallin, alpha B (CRYAB) and family with sequence similarity 5, member C (FAM5C) at a lower degree than mesenchymal stromal cells cultured in a medium supplemented with fetal calf serum.

10. The method of claim 1, wherein the therapeutically effective dose of mesenchymal stromal cells is administered within two to twenty hours after the mammal has received the myeloablative lethal dose of total body irradiation.

11. A therapeutically effective dose of mesenchymal stromal cells for use in treating a mammal which has received a myeloablative lethal dose of total body irradiation.
